(19) United States
(12) Patent Application Publication
(10) Pub. No.: US 2003/0135239 A1
(43) Pub. Date: Jul. 17, 2003

(54) Tissue anchor insertion tool

(76) Inventors: Stefan Gabriel, Mattapoisett, MA (US); Justin Dye, Mansfield, MA (US)

Correspondence Address: JOEL R. PETROW, Smith & Nephew, 1450 Brooks Road, Memphis, TN, 38116, US

(21) Appl. No.: 10/046,290

(22) Filed: Jan. 16, 2002

(51) Int. Cl. A61B 17/04; A61B 17/58

(52) U.S. Cl. 606/232; 606/104

(57) ABSTRACT

A tissue anchor insertion tool includes a first member defining a region configured to receive a tissue anchor, and a second member positioned to substantially cover the tissue anchor during introduction to a surgical site. The second member is coupled to the first member such that relative motion between the members deploys the tissue anchor from the region. The first member includes an applicator configured to move laterally to deploy the anchor from the region. A method includes providing first and second members coupled for relative motion, inserting a tissue anchor into tissue using the first and second members, and relatively moving the first and second members to deploy the tissue anchor from the first member. The tissue anchor is mounted to the first member. The second member substantially covers the tissue anchor during the insertion into tissue.

### BACKGROUND

This invention relates to tissue anchor insertion tools.

Ligaments and tendons, after they have torn away from bone, can be reattached arthroscopically using suture. Traditionally, a surgeon inserts a suture anchor with an attached suture into the bone and ties the suture about the ligament or tendon to secure the ligament or tendon to the bone. The suture anchor is deployed within the bone in a manner that resists pull-out from the bone in response to forces exerted during healing that tend to draw the reattached ligament or tendon, and thus the suture and suture anchor, away from the bone.

### SUMMARY

According to one aspect of the invention, a tissue anchor insertion tool includes a first member defining a region configured to receive a tissue anchor, and a second member positioned to substantially cover the tissue anchor during introduction to a surgical site. The second member is coupled to the first member such that relative motion between the members deploys the tissue anchor from the region.

Embodiments of this aspect of the invention may include one or more of the following features. The first member includes an applicator, and the second member includes a flexor. The members are coupled by engagement of the flexor and the applicator. The applicator includes a straight portion and a ramped portion. The applicator, e.g., a spring, includes a first end portion fixed to the first member and a second end portion extending into the region to engage the tissue anchor. The applicator is configured to move laterally to a direction of relative motion between the members. The flexor includes a pin coupled to the second member for movement with the second member relative to the applicator. The first member defines an opening for receiving the pin.

The first member includes first and second distal prongs defining the region configured to receive a tissue anchor therebetween. The prongs each define arcuate surfaces for receiving the tissue anchor. The second member includes a tubular element substantially surrounding the first member. The tissue anchor insertion tool further includes a contact extending between the first and second members. Actuation of the contact causes relative motion between the first member and the second member. The contact is fixed to the second member. The first member defines a slot for receiving at least a portion of the contact. The tissue anchor insertion tool further includes a handle and a coupling between the handle and the first member preventing relative rotation therebetween.

The tissue anchor insertion tool includes means for applying a lateral force to the tissue anchor. The means includes an applicator and a flexor for flexing the applicator.

According to another aspect of the invention, a tissue anchor insertion tool includes a first member including an applicator and defining a region configured to receive a tissue anchor, and a second member including a flexor and positioned to substantially cover the tissue anchor during introduction to a surgical site. The applicator is configured to move laterally to deploy the tissue anchor from the region. The members are coupled by engagement of the flexor and the applicator such that relative motion between the members causes the applicator to move laterally to deploy the tissue anchor from the region.

According to another aspect of the invention, an anchor and tool assembly includes a tissue anchor, a first member receiving the tissue anchor, and a second member positioned to substantially cover the tissue anchor during introduction to a surgical site and coupled to the first member such that relative motion between the members deploys the tissue anchor from the first member.

According to another aspect of the invention, a tissue anchor insertion tool includes a member defining a region configured to receive a tissue anchor to deliver the tissue anchor to an insertion site. The member includes an applicator configured to move laterally to deploy the anchor from the region configured to receive the tissue anchor.

Embodiments of this aspect of the invention may include one or more of the following features. The tissue anchor insertion tool includes a movable element coupled to the member for movement relative to the member between an extended position and a retracted position. The movable element substantially covers the tissue anchor when in the extended position, and substantially uncovers the tissue anchor when in the retracted position. The movable element includes a flexor coupled to the applicator to laterally move the applicator upon axial movement of the movable element. The applicator includes a straight portion permitting movement of the flexor relative to the applicator without lateral movement of the applicator. The applicator includes a ramped portion, and movement of the flexor along the ramped portion laterally deflects the applicator.

According to another aspect of the invention, a method includes providing first and second members coupled for relative motion, inserting a tissue anchor into tissue using the first and second members, and relatively moving the first and second members to deploy the tissue anchor from the first member. The tissue anchor is mounted to the first member. The second member substantially covers the tissue anchor during the insertion into tissue

Embodiments of this aspect of the invention may include one or more of the following features. The step of relatively moving includes proximally moving the second member relative to the first member. The step of relatively moving uncovers the tissue anchor. Deploying the tissue anchor includes moving an applicator laterally to engage the tissue anchor. Engaging the tissue anchor rotates the tissue anchor.

According to another aspect of the invention, a method includes inserting a tissue anchor into tissue, and moving an applicator laterally to rotate the tissue anchor during deployment of the tissue anchor into tissue.

Embodiments of this aspect of the invention may include covering the tissue anchor during insertion of the tissue anchor into tissue.

The tissue anchor insertion tool enables arthroscopic placement of a tissue anchor that needs to be rotated when implanted. The insertion tool applies a lateral force to the tissue anchor to rotate the anchor, deploying the anchor during an arthroscopic surgical procedure. The tissue anchor is covered during advancement into the tissue, allowing an operator to properly position the insertion tool while limiting the chance of dislodging the tissue anchor from the insertion tool.

The details of one or more embodiments of the invention are set forth in the accompanying drawings and the description below. Other features, objects, and advantages of the invention will be apparent form the description and drawings, and from the claims.

### DESCRIPTION OF DRAWINGS

FIG. 1 is a side view of a tissue anchor insertion tool according to the present invention;

FIG. 2A is an exploded view of the insertion tool;

FIG. 2B is an enlarged view of section 2B of FIG. 2A;

FIG. 3A illustrates a distal end of a cover of the insertion tool;

FIG. 3B is a cross-sectional side view of the cover;

FIG. 4 is a cross-sectional side view of a shaft of the insertion tool;

FIG. 5A illustrates a thumb contact region of the insertion tool;

FIG. 5B is a cross-sectional side view of the thumb contact region of the insertion tool taken along lines 5B-5B of FIG. 5A;

FIG. 6 is a cross-sectional side view of a distal region of the insertion tool;

FIG. 7 illustrates a tissue anchor for use with the insertion tool;

FIGS. 8A-8C are side views of the insertion tool shown at various stages during deployment of the tissue anchor; and

FIGS. 9A and 9B illustrate deployment of the tissue anchor in bone.

### DETAILED DESCRIPTION

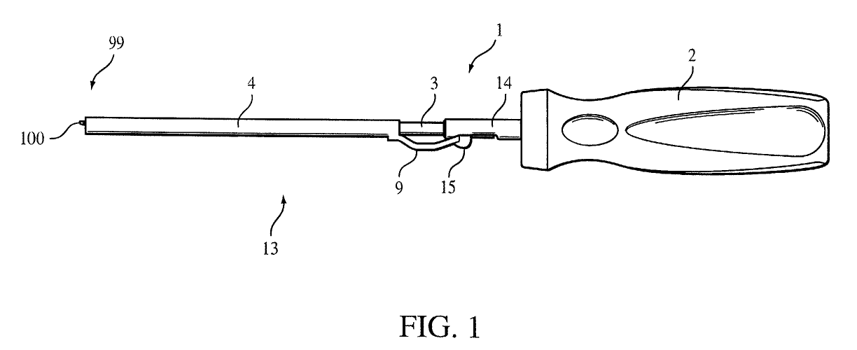

Referring to FIG. 1, an anchor insertion tool 1 arthroscopically deploys a tissue anchor 100, e.g., the RotorloC™ Anchor available from Smith & Nephew Endoscopy, Andover, Mass., by axially advancing the anchor into a bone hole and applying a lateral force to the anchor to rotate the anchor. Tool 1 includes a handle 2 joined to an elongate portion 13 terminating in a distal region 99 housing tissue anchor 100. Elongate portion 13 includes an adapter 14 that is coupled to handle 2, a shaft 3 coupled to adapter 14, and a tubular cover 4 surrounding shaft 3. Cover 4 is coupled to adapter 14 to slide relative to adapter 14, as described below. Tissue anchor 100 is located within shaft 3 and substantially covered by cover 4 during introduction to a surgical site.

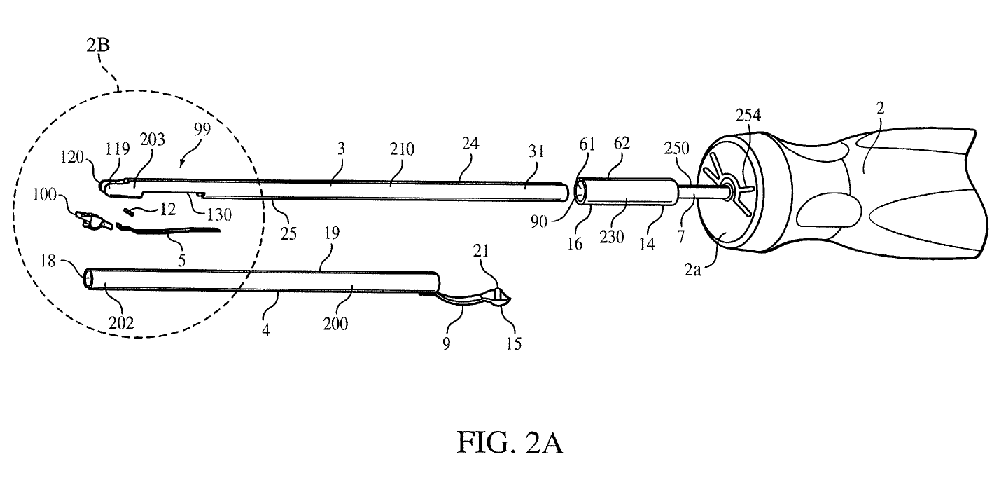

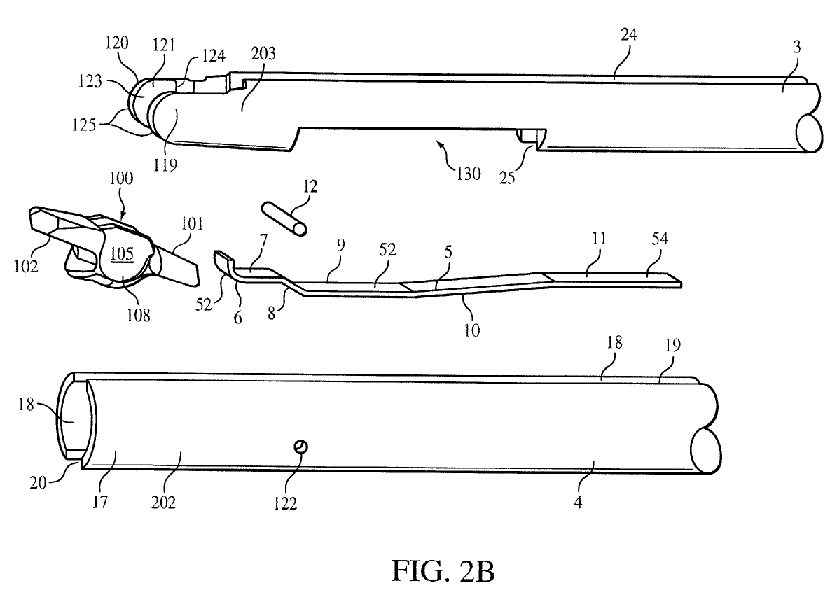

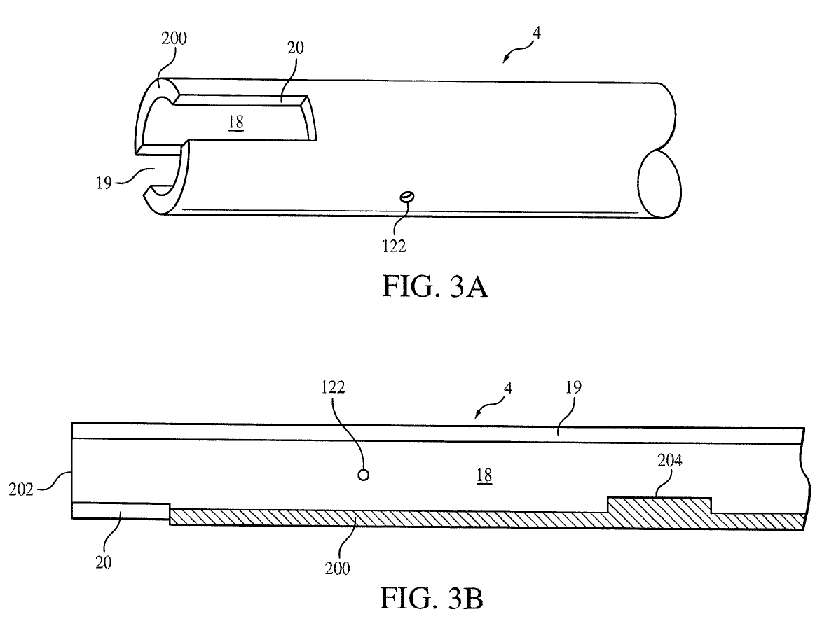

Referring to FIGS. 2A, 3A and 3B, cover 4 is a tubular member having a wall 200 defining a lumen 18 for receiving shaft 3, and a slot 19 extending through wall 200 along the entire length of cover 4. Opposite slot 19, an additional slot 20 extends through wall 200 over a length of about 5 to 15 mm, preferably about 10 mm, from a distal end 202 of cover 4, for purposes described below. Extending from wall 200 into lumen 18 is a guide 204.

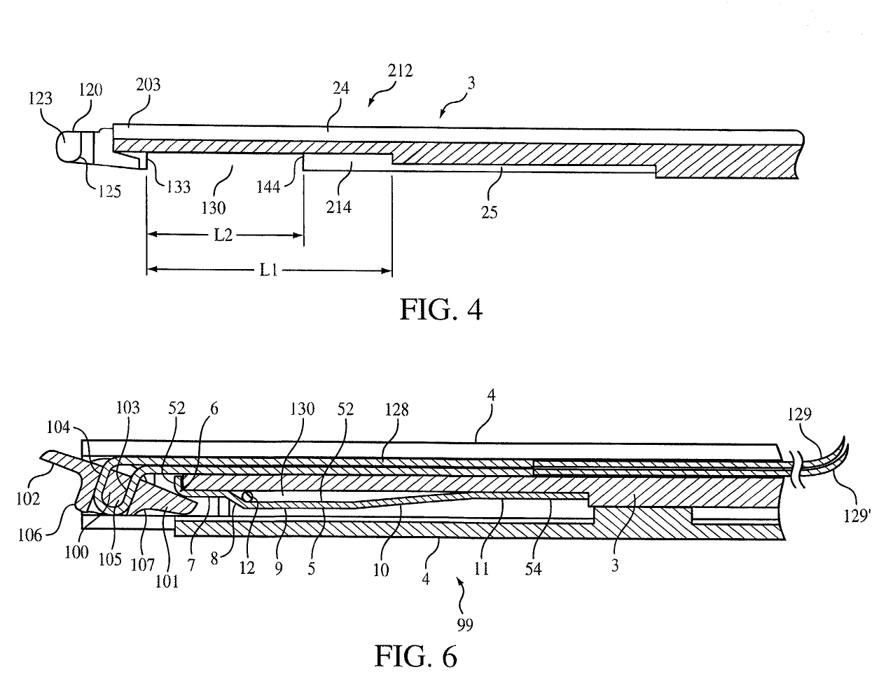

Referring to FIGS. 2A and 4, shaft 3 is a solid member with a first slot 24 in an exterior surface 210 of the shaft, and an opposite slot 25 in the exterior surface 210 of the shaft. Slot 24 extends the entire length of shaft 3. Guide 204 is received within shaft slot 25 limiting relative rotation between shaft 3 and cover 4 while allowing relative axial motion. Slot 25 extends up to about 150 mm, preferably about 95 mm, from distal end 203 of shaft 3, and guide 204 is spaced about 100 mm, preferably about 65 mm, from distal end 202. The relative length of slot 25 and positioning of guide 204 provides clearance for a desired amount of relative axial motion between shaft 3 and cover 4.

The depth of shaft slot 25 is increased in a distal region 212 of shaft 3 over a length L1 of about 20 to 50 mm, preferably about 35 mm to form a chamber 214, for purposes described below. The width of shaft slot 25 is increased in distal region 212 of the shaft over a length L2 of about 10 to 30 mm, preferably about 20 mm, to form a cutout 130 having distal and proximal ends 133, 144, respectively, for purposes described below.

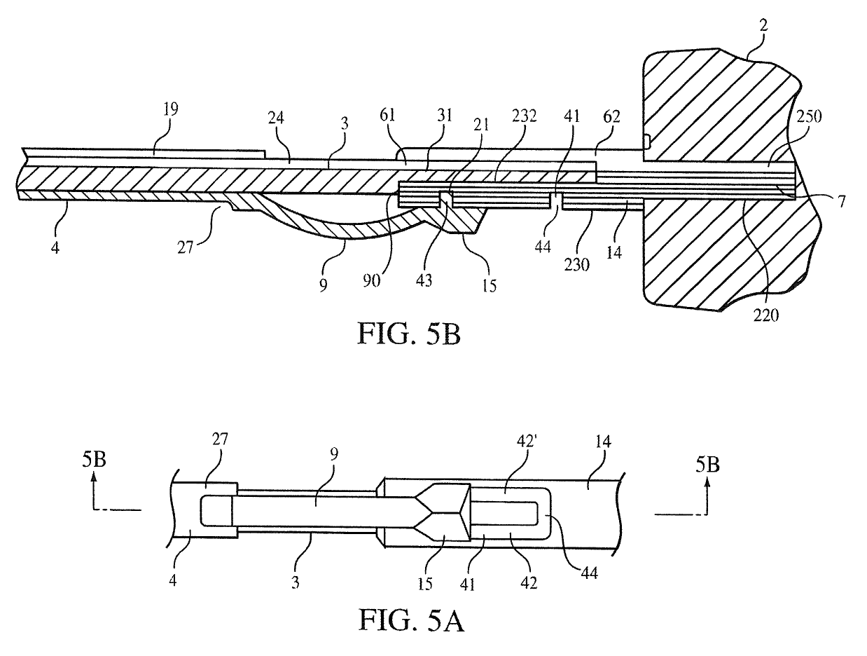

Referring to FIGS. 2A and 5B, adapter 14 includes a coupling portion 7 received within a bore 220 in handle 2 and fixed to handle 2 by, e.g., epoxy. Coupling portion 7 defines a slot 250. Adapter 14 has a wall 230 defining a bore 61 and a slot 62 extending from bore 61 through wall 230. Slot 62 is aligned with slot 250. Opposite slot 62, an axial nub 90 extends from wall 230 into bore 61 and runs the length of adapter 14. Shaft 3 has an additional slot 232 opposite slot 24 that receives nub 90 when the proximal end 31 of shaft 3 is slid into bore 61. The placement of nub 90 within slot 232 limits relative rotation between shaft 3 and adapter 14.

Referring to FIGS. 5A and 5B, cover 4 is coupled to adaptor 14 by a resilient thumb contact 9. Contact 9 extends from a proximal end 27 of cover 4 to a guide channel 41 defined in adapter 14. Contact 9 includes a mating member 15 supporting a nub 21 that is received in guide channel 41. Guide channel 41 has a race-track shape with proximal and distal portions 43, 44, respectively, and side portions 42, 42′. In an unstressed state, contact 9 is straight with nub 21 in the middle of portion 43 or 44. To axially move cover 4, the operator flexes contact 9 sideways to align nub 21 with side portion 42 or 42′ and moves nub 21 axially along side portion 42 or 42′. When nub 21 has been moved the full length of the side portion, contact 9 springs back to a straight orientation returning nub 21 to the middle of portion 43 or 44. This spring action provides positive control on the relative motion between cover 4 and shaft 3. The distance between proximal and distal portions 43, 44 is, e.g., about 10 to 20 mm, preferably about 15 mm, and defines the range over which cover 4 can be slid relative to shaft 3.

Referring to FIGS. 2B and 6, cover 4 includes a flexor, e.g., a pin 12, and shaft 3 includes an applicator, e.g., a spring 5 located within chamber 214. Pin 12 and spring 5 couple cover 4 and shaft 3 such that retraction of cover 4 relative to shaft 3 causes lateral deflection of spring 5, as described below. Spring 5 is received within shaft chamber 214 and has a proximal end 54 attached to shaft 3 by e.g., epoxy, and a free distal end 51. Cover 4 defines a pair of opposing holes 122 in which pin 12 is received such that pin 12 extends through lumen 18. As shown in FIG. 6, pin 12 is received within cutout 130 between shaft 3 and spring 5 and contacts a surface 52 of spring 5. The length of cutout 130 provides clearance for desired axial motion of pin 12.

Spring 5 is contoured to control lateral flexing of spring 5 as pin 12 is moved along surface 52 of spring 5. From distal end 51 to proximal end 54, spring 5 includes an arcuate portion 6 that engages anchor 100, a straight portion 7, a sloped portion 8, a straight portion 9, a sloped portion 10, and a straight portion 11. When cover 4 is moved relative to shaft 3, pin 12 slides along surface 52 of spring 5. When pin 12 engages portion 10 of spring 5, spring 5 deflects laterally, moving distal end 51 of spring 5 laterally against anchor 100 to deploy anchor 100 from tool 1, as described further below.

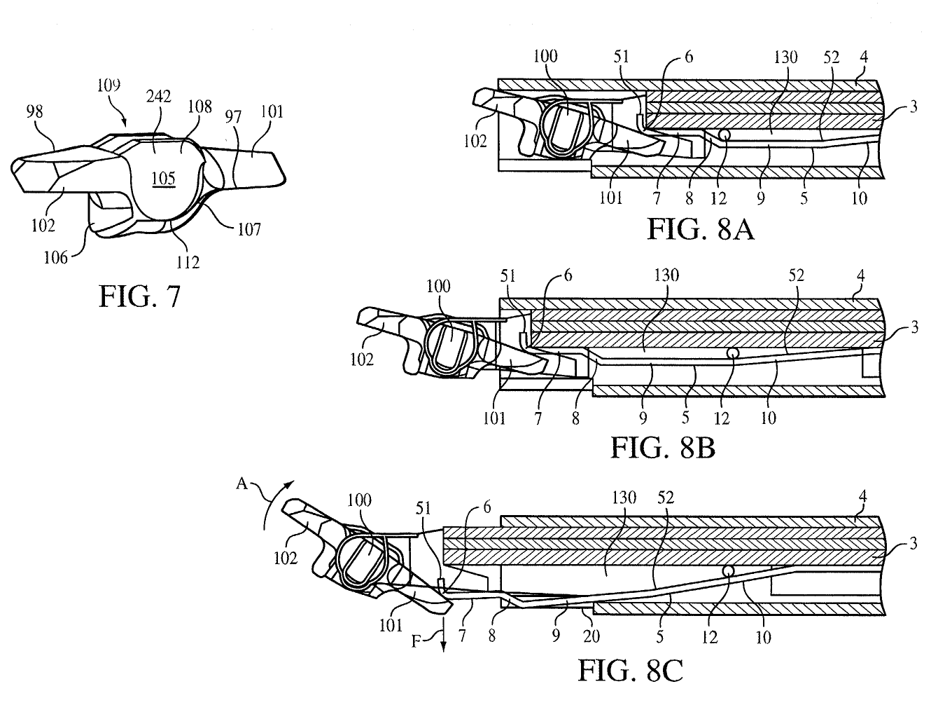

Shaft 3 includes a pair of opposing, spaced apart arms 119, 120 that define an anchor receiving region 121 therebetween. Each arm 119, 120 has an internal pivot face 123 bounded by an arcuate edge 125. Tissue anchor 100 is coupled to shaft 3 by placement between arms 119, 120 in abutment with faces 123. Free end 54 of spring 5 extends into region 121 and contacts anchor 100. Referring also to FIG. 7, tissue anchor 100 includes a central portion 105 with an opposing pair of pivoting faces 108, 109. Each pivoting face 108, 109 includes a raised arcuate lip 112 with a radius of curvature substantially equal to the radius of curvature of arcuate edges 125 of arms 119, 120. When assembled, faces 123 of arms 119, 120 are positioned against anchor faces 108, 109, with edges 125 against lips 112. Due to the shapes of edges 125 and lips 112, anchor 100 can rotate relative to arms 119, 120. Lip 112 does not define a complete circle about faces 108, 109 such that anchor 100 has an opening 242 to each of faces 108, 109. When anchor 100 is slid between arms 119, 120, arm pivot faces 123 pass through openings 242 into position against anchor faces 108, 109. Anchor 100 is maintained in position between arms 119, 120 by the engagement of lips 112 with edges 125, and by the positioning of cover 4 about anchor 100.

Tissue anchor 100 includes a pair of wings 101, 102 with oppositely oriented, angled cutting edges 97, 98, respectively. As shown in FIG. 6, central portion 105 of tissue anchor 100 defines a pair of suture channels 103, 104 for receiving two suture strands 128 (only one suture strand being shown). When assembled, with suture strands 128 threaded through channels 103, 104, suture 128 passes between arms 119, 120 to slot 24, and along slot 24 to adapter slot 250. At the end of each suture strand there is a needle 129, 129′. Handle 2 has a face 2a defining four slots 254 (FIG. 2A) in which the needles are located during introduction of anchor 100 into tissue.

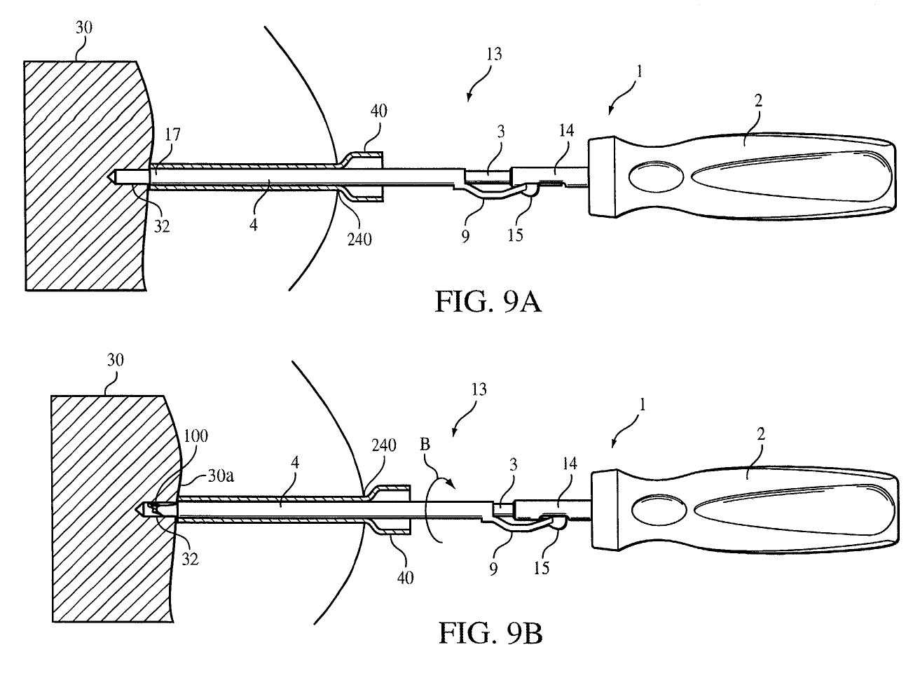

Referring to FIGS. 8A and 9A, during introduction of tool 1 into tissue, mating member 15 is in contact with distal face 44 of guide channel 41 and pin 12 is near distal end 133 of cutout 130 such that cover 4 is disposed distally to substantially cover tissue anchor 100. Spring 5 rests against tissue anchor 100 without exerting a lateral force on the anchor, and pin 12 contacts face 52 of spring 5 at the junction of spring portions 8 and 9. The position of cover 4 over anchor 100 limits possible dislodgement of anchor 100 from tool 1 during introduction into the tissue, and protects the tissue from the anchor.

Referring to FIG. 8B, to deploy the anchor, the operator first slides member 15 and thus cover 4 proximally to a position near the middle of the slidable range (i.e., member 15 is near the middle of guide channel 41 and pin 12 is near the middle of cutout 130). Pin 12 now contacts spring 5 at the junction of spring portions 9 and 10, and anchor 100 is partially uncovered. Since spring portion 9 is oriented parallel to the axis of elongate portion 13, the movement of pin 12 does not deflect spring 5 and spring 5 still rests against tissue anchor 100 without exerting a lateral force on the anchor.

Referring to FIGS. 8C and 9B, to rotate anchor 100 (arrow A), the operator slides member 15 and thus cover 4 further proximally such that member 15 is in contact with proximal face 43 of guide channel 41 and pin 12 is near distal end 144 of cutout 130. Anchor 100 is now fully uncovered. The movement of pin 12 along sloped spring portion 10 laterally deflects spring 5. Spring portions 8 and 9 are received within cover slot 20, and distal spring portion 6 exerts a substantially laterally directed force, F, on anchor 100 causing the anchor to rotate. The rotation of tissue anchor 100 pivots anchor 100 within arms 119, 120. The proximal translation of cover 4 thus both exposes and rotates anchor 100.

Referring to FIGS. 9A and 9B, in use, e.g., in shoulder repair, with a cannula 40 placed through a skin portal 240, the operator advances tissue anchor insertion tool 1 through cannula 40 to a predrilled hole 32 in a tissue 30, e.g., bone tissue. The operator then moves member 15 proximally to channel portion 44, thus moving cover 4 proximally, while pushing insertion tool 1 into hole 30. This results in shaft 3 entering the bone hole with the distal end of cover 4 abutting the bone surface 30a, and anchor 100 is uncovered and rotated, as described above, with the ends of tissue anchor wings 101, 102 starting to push into the bone tissue surrounding hole 32. The operator then applies a torque to handle 2 to rotate insertion tool 1 and tissue anchor 100, arrow B. The applied torque causes edges 97, 98 of anchor 100 to cut into the bone tissue, and, because the cutting edges are set at an angle, the rotation of anchor 100 along arrow B results in additional rotation of anchor 100 along arrow A. About 1½ turns of tool 1 rotates anchor 100 such that anchor wings 101, 102 are embedded in the bone tissue and oriented substantially perpendicular to the bone wall. The rotation of anchor 100 to this perpendicular position aligns anchor face openings 242 with arms 119, 120 such that arms 119, 120 can be slid from anchor 100 through openings 242. Thus, to release anchor 100 from shaft 3, the operator simply moves tool 1 proximally.

Other embodiments are within the scope of the following claims.

What is claimed

1. A tissue anchor insertion tool comprising:
a first member defining a region configured to receive a tissue anchor; and
a second member positioned to substantially cover the tissue anchor during introduction to a surgical site and coupled to the first member such that relative motion between the members deploys the tissue anchor from the region.

2. The tissue anchor insertion tool of claim 1 wherein the first member includes an applicator, and the second member includes a flexor, the members being coupled by engagement of the flexor and the applicator.

3. The tissue anchor insertion tool of claim 2 wherein the applicator includes a straight portion and a ramped portion.

4. The tissue anchor insertion tool of claim 2 wherein the applicator includes a first end portion fixed to the first member, and a second end portion extending into the region to engage the tissue anchor.

5. The tissue anchor insertion tool of claim 2 wherein the applicator comprises a spring.

6. The tissue anchor insertion tool of claim 2 wherein the applicator is configured to move laterally to a direction of relative motion between the members.

7. The tissue anchor insertion tool of claim 2 wherein the flexor comprises a pin coupled to the second member for movement therewith relative to the applicator.

8. The tissue anchor of claim 7 wherein the first member defines an opening for receiving the pin.

9. The tissue anchor insertion tool of claim 1 wherein the first member includes first and second distal prongs defining the region therebetween.

10. The tissue anchor insertion tool of claim 9 wherein the prongs each define arcuate surfaces for receiving the tissue anchor.

11. The tissue anchor insertion tool of claim 1 wherein the second member comprises a tubular element substantially surrounding the first member.

12. The tissue anchor insertion tool of claim 1 further comprising a contact extending between the first and second members, actuation of the contact causing relative motion between the first member and the second member.

13. The tissue anchor insertion tool of claim 12 wherein the contact is fixed to the second member.

14. The tissue anchor insertion tool of claim 13 wherein the first member defines a slot for receiving at least a portion of the contact.

15. The tissue anchor insertion tool of claim 1 further comprising a handle.

16. The tissue anchor insertion tool of claim 14 further comprising a coupling between the handle and the first member preventing relative rotation therebetween.

17. The tissue anchor insertion tool of claim 1 further including means for applying a lateral force to the tissue anchor.

18. The tissue anchor insertion tool of claim 17 wherein the means includes an applicator and a flexor for flexing the applicator.

19. A tissue anchor insertion tool comprising:
a first member including an applicator and defining a region configured to receive a tissue anchor, the applicator configured to move laterally to deploy the tissue anchor from the region;
a second member including a flexor and positioned to substantially cover the tissue anchor during introduction to a surgical site, the members being coupled by engagement of the flexor and the applicator such that relative motion between the members causes the applicator to move laterally to deploy the tissue anchor from the region.

20. An anchor and tool assembly, comprising:
a tissue anchor;
a first member receiving the tissue anchor; and
a second member positioned to substantially cover the tissue anchor during introduction to a surgical site and coupled to the first member such that relative motion between the members deploys the tissue anchor from the first member.

21. A tissue anchor insertion tool comprising:
a member defining a region configured to receive a tissue anchor to deliver the tissue anchor to an insertion site; the member including an applicator configured to move laterally to deploy the anchor from the region.

22. The tissue anchor insertion tool of claim 21 further comprising a movable element coupled to the member for movement relative to the member between an extended position and a retracted position, the movable element substantially covering the tissue anchor when in the extended position, and substantially uncovering the tissue anchor when in the retracted position.

23. The tissue anchor insertion tool of claim 22 wherein the movable element includes a flexor coupled to the applicator to laterally move the application upon axial movement of the movable element.

24. The tissue anchor insertion tool of claim 23 wherein the applicator includes a straight portion permitting movement of the flexor relative to the applicator without lateral movement of the applicator.

25. The tissue anchor insertion tool of claim 23 wherein the applicator includes a ramped portion, wherein movement of the flexor along the ramped portion laterally deflects the applicator.

26. A method comprising:
providing an insertion tool including first and second members coupled for relative motion;
inserting a tissue anchor into tissue using the insertion tool, the tissue anchor being mounted to the first member and substantially covered by the second member during insertion into tissue; and
relatively moving the first and second members to deploy the tissue anchor from the first member.

27. The method of claim 26 wherein the step of relatively moving comprises proximally moving the second member relative to the first member.

28. The method of claim 26 wherein the step of relatively moving uncovers the tissue anchor.

29. The method of claim 26 wherein the step of relatively moving deploys the tissue anchor by moving an applicator laterally to engage the tissue anchor.

30. The method of claim 29 wherein engaging the tissue anchor rotates the tissue anchor.

31. An arthroscopic method comprising:
inserting a tissue anchor into tissue; and
moving an applicator laterally to rotate the tissue anchor during deployment of the tissue anchor into tissue.

32. The method of claim 31 further comprising substantially covering the tissue anchor during insertion of the tissue anchor into tissue.
